(19) United States
(12) Patent Application Publication
(10) Pub. No.: US 2017/0333688 A1
(43) Pub. Date: Nov. 23, 2017

(54) DISPESNING DEVICES FOR TOPICAL PHARMACEUCTAL COMPOSITIONS

(71) Applicants: Nilesh Parikh, Irvine, CA (US); William C Hite, Winchester, CA (US)

(72) Inventors: Nilesh Parikh, Irvine, CA (US); William C Hite, Winchester, CA (US)

(21) Appl. No.: 15/526,311

(22) PCT Filed: Nov. 11, 2015
(86) PCT No.: PCT/US15/60152

§ 371 Date: May 11, 2017

Related U.S. Application Data
(60) Provisional application No. 62/079,112, filed on Nov. 13, 2014.

(51) Int. Cl. A61M 35/00; A61K 31/565; A45D 40/26; A61K 9/00; A61K 31/568; A45D 40/04; A61K 9/06; A61Q 15/00; A61K 8/04

(52) U.S. Cl. CPC A61M 35/003; A61Q 15/00; A61K 31/565; A45D 40/261; A61K 8/042; A61K 31/568; A45D 40/04; A61K 9/06; A61K 9/0014; A61K 2800/48; A61M 2205/103; A61M 2205/8206

(57) ABSTRACT

The present invention provides devices for dispensing topical pharmaceutical compositions. In some embodiments, the dispensing device possesses a reservoir for storing the composition and means for metering the composition. In some embodiments, such reservoirs are: i. made from, or substantially lined with, a material that opaque and/or substantially nondepleting of at least one component of the composition; ii. optionally, volumetrically compressible; and iii. fitted with the means for metering the composition. And the means for metering are adapted to measure a fixed amount of the composition for application to a dermal region of a user of the device. In some embodiments, the compositions has a specific gravity in the range of from about 0.85 to about 1.20 at about room temperature and comprises a medicament; a thickening agent; and a solvent, and optionally a skin penetration enhancer and/or a deodorant.

The present application claims the benefit of International Patent Application PCT PCT US201560152, filed on Nov. 11, 2015 and U.S. Provisional Patent Application Ser. No. 62/079,112, filed on Nov. 13, 2014, each of which are hereby incorporated by reference in its entirety.

### FIELD OF THE INVENTIONS

The present invention relates to devices adapted for containing and dispensing topically-administered, nonpourable, pharmaceutical compositions, such as gels, creams, ointments, pastes, or lotions, to skin and/or mucosa of a mammal, e.g., a human.

### BACKGROUND OF THE INVENTIONS

The topical administration of active pharmaceutical ingredients (APIs) offers several potential benefits. For instance, sites for the topical application of APIs, such as skin or mucosa, can be easily accessed by patients; and therefore allow for efficient administration. And such sites typically afford comfortable application of topical compositions, which promotes patient compliance with treatment regimens.

The topical delivery of APIs offers the potential for the targeted treatment of conditions that are local, either in causality or manifestation, with relatively high local concentrations of APIs; and therefore can both generally increase efficacy and, for APIs having toxicity or irritation potential, reduce side effects reduce. Topical administration of APIs further offers the potential for the effective treatment of systemic conditions because of the significant exposure of sites for the topical administration of APIs to the circulatory system. The availability of skin penetration or permeability enhancers for use in topical, pharmaceutical compositions can facilitate the formulation of certain APIs possessing relatively low skin permeation properties for systemic effect.

Other potential advantages of topically applied pharmaceutical formulations include avoidance of drawbacks associated with parenteral and/or oral dosage forms. Such drawbacks spanning risks of variable adsorption and/or metabolism of APIs; noncontinuity of target tissue exposure to APIs (e.g., for APIs having short biological half-lives); insufficient exposure of target tissue to APIs (e.g., for APIs having limited bioavailability/access to target tissue; and inconvenient, irritating, or painful administration.

### SUMMARY OF THE INVENTIONS

Prescription therapy of mental or physical condition, illness, or disease involves a health care professional selecting for administration a drug having curative or palliative effect. And prescription drug therapy requires, for achieving efficacy and minimizing side effects, accurate drug dosing, in terms of both amount and interval. Pharmaceutical dosage forms, such as tablets, capsules, powders, and liquids, which are disposed to unit dose administration of precisely calibrated amounts of drug afford accurate dosing.

Precision dosing is more difficult with topical pharmaceutical dosage forms, such as gels, creams, ointments, and lotions, which are not disposed to unit dose administration of precisely calibrated amounts of drug, due to variety of causations. For instance, topical pharmaceutical dosage forms, such as gels, creams, ointments, and pastes, are commonly packaged in tubes that have no means for measuring the amount of the formulation dispensed therefrom and subsequently administered by finger or hand to the target site of application of a patient. Such that the dispensed amount of formulation is frequently not the prescribed amount. In addition, some of the formulation is absorbed by the skin of the finger or hand of the administrator such that the dispensed amount is not the amount applied to the target site. The latter mechanism for inaccurate dosing of topical pharmaceutical compositions occurs even when they are dispensed in measured amounts, such as by pump or removal from a preloaded unit dose packet.

Another source of inaccurate dosing of topical pharmaceutical compositions is the absorption of drug or other excipient, such as a penetration or a permeation enhancer, by the material of the dispensing unit's reservoir for the composition. Such absorption commonly occurs slowly, and removes drug or excipient from the composition over time. Thereby reducing the actual or effective amount of drug administered to the target tissue. Such reductions can also result, in the context of light sensitive drugs or excipients, from the construction of a dispensing unit's reservoir with transparent or translucent material allowing for the photolytic degradation of such drugs or excipients over time. Or from the construction of the dispensing unit's reservoir with materials that leach drug or excipient degrading chemicals into the composition.

Hand or finger administration of topical, pharmaceutical compositions has the potential for other problems. When administrators fail to take sufficient measures to clean residual formulation from their hand or finger post application, the residual formulation can be efficiently transferred to unintended persons by touch, either directly or indirectly. And such transfer can cause toxicity and other unwanted effects. Especially in the contexts of the transfer of potent drugs, such as hormones or steroids, and sensitive transferees, such as children or pregnant women. Although the problem of unintended transfer can be solved by the administrator wearing a latex glove during application, this solution is objectionable on many grounds. Many people find the feel of latex gloves unacceptable and uncomfortable. Also, many people are allergic to latex, and their use of latex gloves can cause anaphylactic shock. Further, latex gloves are an additional expense to drug therapy.

There exists a need in the art for the development of a dispensing device and method for topical pharmaceutical compositions which addresses inaccurate dosing and other problems discussed above. Accordingly, embodiments of the present invention provide devices for metered dispensing of topical pharmaceutical compositions. In some embodiments, the dispensing device possesses a reservoir for storing the composition and means for metering the composition. In some embodiments, such reservoirs are: i. made from, or substantially lined with, a material that is opaque and/or substantially nondepleting of at least one component of the composition; ii. optionally, volumetrically compressible; and iii. fitted with the means for metering the composition. And the means for metering are adapted to measure a fixed amount of the composition for application to a dermal region of a user of the device. In some embodiments, the metered compositions have a specific gravity of from about 0.85 to about 1.20, preferably about 0.90 to about 1.15 at about room temperature and comprise a medicament; a thickening agent; and a solvent, and optionally a skin penetration enhancer, an antiperspirant, and/or a deodorant.

### BRIEF DESCRIPTION OF THE DRAWINGS

FIG. 1 is a front view of an embodiment of a dispenser device according to the invention that possesses a dial actuator and an electronic dose indicator, with side wall cutouts indicated by dashed lines. FIG. 1A shows the dispensing device after one use, with cap off. FIG. 1B shows the same dispensing device after two actuations, but prior to second application. FIG. 1C shows the same dispensing device after two uses, with cap on. FIG. 1D is a cutout view of the cap for the dispenser device that possesses pins that seal application surface apertures and an O-ring that seals the cap to the sidewalls during storage of the device.

FIG. 2 is a cross sectional view of a cap, for a dispenser device according to the invention, fitted with locking mechanism. FIG. 2A illustrates the cap in the lock position. FIG. 2B illustrates the cap in the release position.

FIG. 3 is a front view of an embodiment of a dispenser device according to the invention that possesses a slot and key actuator, with side wall cutouts indicated by dashed lines. FIG. 3A illustrates the dispenser device with key-cap secured in a closed position. FIG. 3B illustrates the dispenser device with key-cap positioned with the key friction fitted into its receiving slot. FIG. 3C illustrates the dispenser after one advancing actuation executed by a 90° rotation of key-cap.

FIG. 4 is a front view of an embodiment of a dispenser device according to the invention that possesses a battery operated peristaltic pump, with side wall cutouts indicated by dashed lines. FIG. 4A shows the dispensing device prior to one use. FIG. 4B shows the same dispensing device after one actuation, but prior to application.

FIG. 5 is a front view of an embodiment of a dispenser device according to the invention that possesses a crank operated peristaltic pump, with side wall cutouts indicated by dashed lines. FIG. 5A shows the dispensing device prior to one use. FIG. 5B shows the dispensing device prior to one use, with the slot key of its removable cap positioned in the actuator slot. FIG. 5C shows the dispensing device after one actuation, but prior to application.

FIG. 6 is a front view of an embodiment of a squeezable dispenser device according to the invention that possesses a roller ball applicator, with side wall cutouts indicated by dashed lines. FIG. 6A shows the dispensing device prior to one use. FIG. 6B shows the dispensing device actuated by compression.

FIG. 7 is a front view of an embodiment of a dispenser device according to the invention that possesses an actuator dial and a roller bar applicator, with side wall cutouts indicated by dashed lines. FIG. 7A shows the dispensing device prior to one use. FIG. 7B shows the dispensing device after actuation, but prior to application.

FIG. 8 is a front view of an embodiment of a dispenser device according to the invention that possesses a dial actuator and a mechanical dose indicator, with side wall cutouts indicated by dashed lines. FIG. 8A shows the dispensing device after one use. FIG. 8B shows the same dispensing device after two actuations, but prior to second application.

### DETAILED DESCRIPTION OF THE INVENTIONS

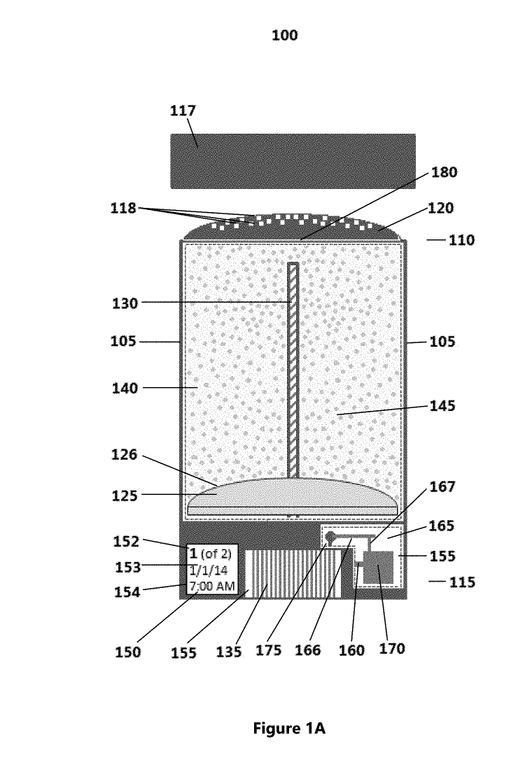

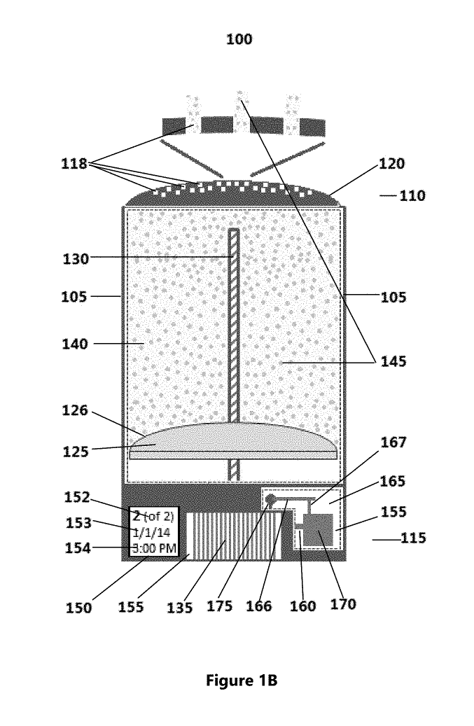

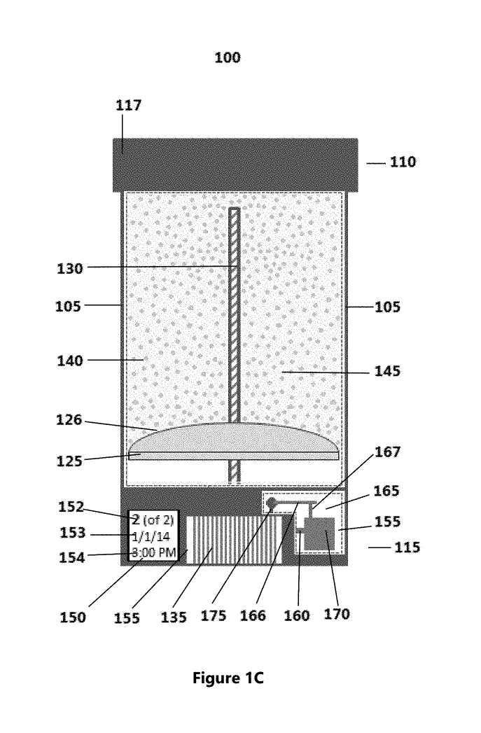

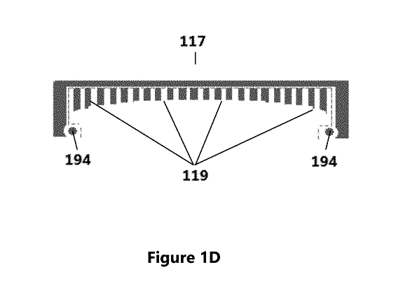

Devices. FIG. 1A illustrates a front view of an embodiment of a semi-solid, topical, pharmaceutical composition dispensing device 100 according to the present invention, with sidewall cutouts indicated by dashed lines. Dispenser device 100 possesses sidewalls 105, application end 110, dispensing end 115, and removable cap 117. Dispenser device 100 is sized and shaped for facile and comfortable hand held use. Sidewalls 105 are integral with each of application end 110 and dispensing end 115; and removable cap 117 is sized and shaped for frictional securement with the sidewalls 105 at application end 110. Application end 110 comprises convex, apertured 118 application surface 120. Disposed within sidewalls 105 at dispensing end 115 is a dispensing assembly that includes platform 125, threaded shaft 130, and actuator dial 135. Disposed within sidewalls 105, application surface 120, and platform 125, is volumetrically collapsible reservoir 140 loaded with semi-solid, topical, pharmaceutical composition 145 that has a specific gravity of from about 0.85 to about 1.20, preferably about 0.90 to about 1.15 at about room temperature. Cap 117 possesses pins 119 that are arrayed and shaped such that they align with and plug dispensing surface apertures 118 when cap 117 is secured on device 100. Cap 117 possesses O-ring 194 that forms a seal between sidewalls 105 and cap 117 (FIG. 1D). Actuator dial 135 is touch accessible through opening 155 in sidewalls 105.

Platform 125 has convex surface 126 and is in circumferentially wiping contact with inner surfaces of sidewalls 105. Platform 125 is threadedly secured to drive shaft 130, which is fixedly secured to actuator dial 135 substantially at the center thereof. The thread pitch of threaded shaft 130 is selected in conjunction with the diameter of actuator dial 135, the size of the apertures 118, and the specific gravity of pharmaceutical composition 145 so that one advancing action of actuator dial 135 (to produce a shaft 130 rotation of from about 10° to about 150°) advances platform 125 a distance toward application surface 120 sufficient to result in a particular volume of pharmaceutical composition 145, ranging from about 50 microliters to about 15,000 microliters, being extruded through apertures 118 (FIG. 1B). Accordingly, rubbing application surface 120 against skin after one, two, three, etc. advancing actions transfers to the skin a metered amount of extruded pharmaceutical composition 145. After application, cap 117 is secured to dispensing end 115 for protective storage until the next application is desired (FIG. 1C).

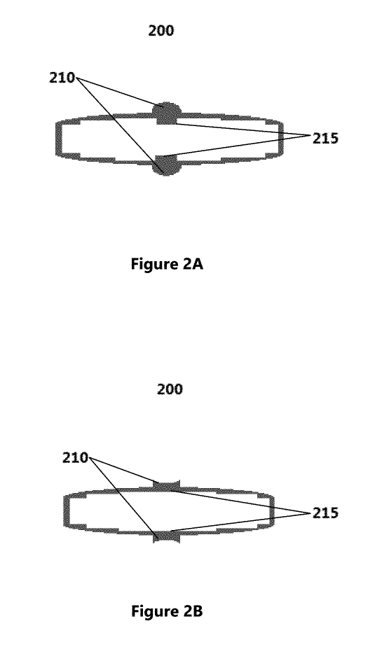

In some embodiments, caps for dispensers are equipped with safety locks that inhibit unwanted (e.g., by child or accident) cap removal. FIGS. 2A and 2B illustrate cap 200, which differs from cap 117 by way of being equipped with locking mechanisms made up of push button release units 210 and tongue lock members 215. FIG. 2A illustrates cap 200 with locking mechanism in the locked position. In the locked position, push button release units 210 are in the illustrated uncompressed position. Tongue lock members 215 are sized and positioned on cap 200 such that, in the locked position, tongue lock members 215 protrude into tongue lock receiving member groove 180 on dispensing device 100 (see FIG. 1A).

FIG. 2B illustrates cap 200 with locking mechanism 205 in the released position. In the released position, push button release units 210 are in the illustrated compressed position, which causes the retraction of tongue lock members 215 from lock receiving member groove 180 and allows for the removal of cap 200.

Referring again to FIGS. 1A-1C The dispenser device 100 further possesses electronic a dose count display unit that comprises screen 150 in electronic communication with dose counter mechanism 155 via wire harness 160. Screen 150 possesses readout lines for the date 152, time 153, and dose number of total daily doses 154 dispensed by device 100. Dose counter mechanism 155 comprises pivot switch 165 and mechanism housing 170 fixedly mounted on sidewall 105. Fixedly mounted on actuator dial 135 are a plurality of ball and stick dose counter triggers 175 (one shown). Dose counter triggers 175 are positioned along the circumference of a circle that is concentric with, and has a smaller radius than, actuator dial 135.

Pivot switch 165 comprises deflection bar 166 fixedly secured to the top of switch pin 167, which is rotatably mounted into the housing of douse counter mechanism 170. Pivot switch 165 is tension mounted in the illustrated position. Toward the end of one advancing action of actuator dial 135, movement of a dose counter trigger 175 along its arced path, caused by finger advancing actuator dial 135, results in forceful contact by the ball portion of dose counter trigger 175 against deflection bar 166. Such contact causes deflection bar 166 to deflect out of the path of dose counter trigger 175, which in turn causes axial rotation of switch pin 167. At the end of one advancing action of actuator dial 135, dose counter trigger 175 moves further along its arced path out of contact with deflection bar 166, which allows pivot switch 165 to return to its tension mounted position. At the point of maximal rotation of switch pin 167, achieved at the point of maximum deflection of deflection bar 166 away from its tension mounted position, the rotation of switch pin 166 causes the transmission of an electronic signal that results in dose counter display unit 150 updating readout lines 152-154 to reflect and describe the occurrence of an actuation event.

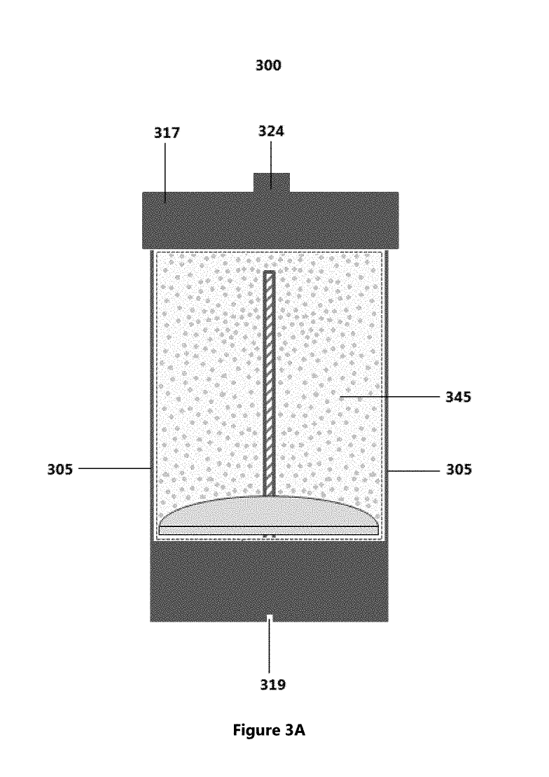

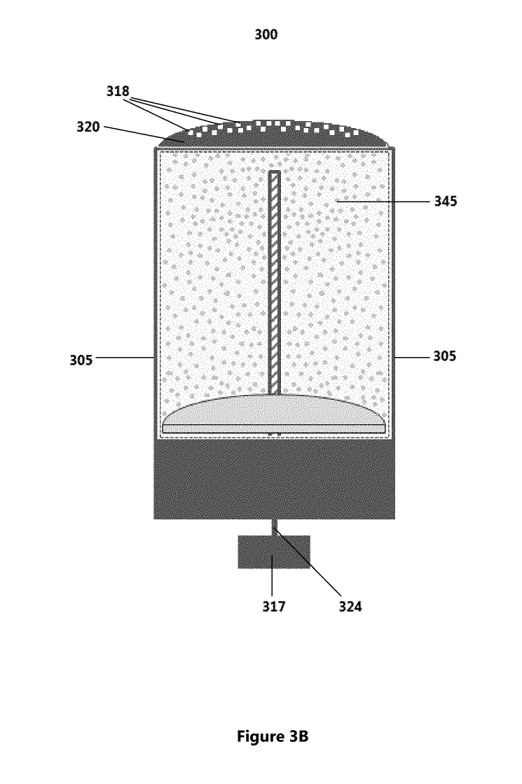

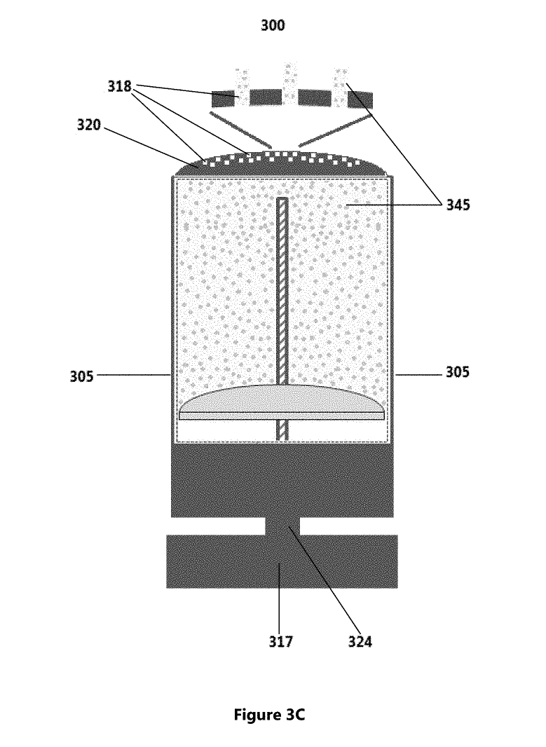

FIG. 3 illustrates a front view of a dispenser device 300 that differs from the device illustrated in FIG. 1 by lacking a dose count indicator and possessing a slot and key actuator rather than an actuator dial, with sidewall cutouts indicated by dashed lines. FIG. 3A illustrates dispenser device 300 with key-cap 317 secured in a closed position. Cap 317 possesses key 324 that is sized and shaped for friction fitment into slot 319 positioned substantially at the center of actuator dial 335. FIG. 3B illustrates dispenser device 300 having key-cap 317 positioned with key 318 friction fitted into slot 319. FIG. 3C illustrates the dispenser device 300 shown in FIG. 3B after one advancing actuation executed by a 90° rotation of key-cap 317.

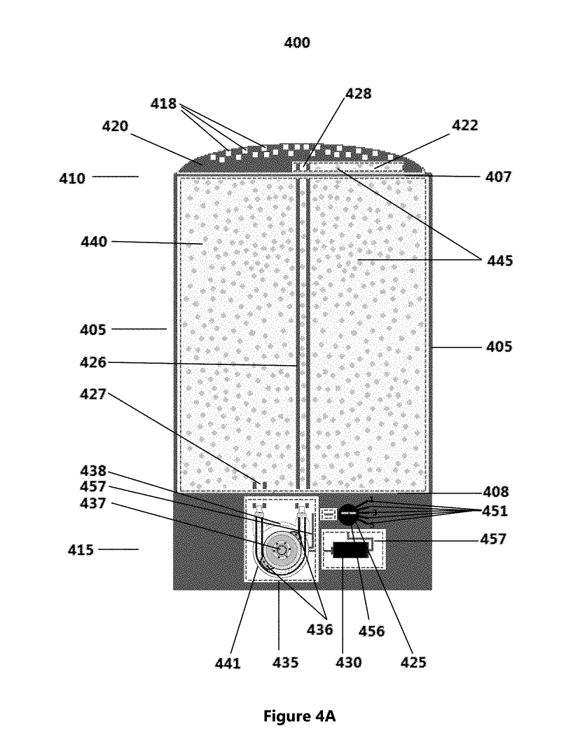

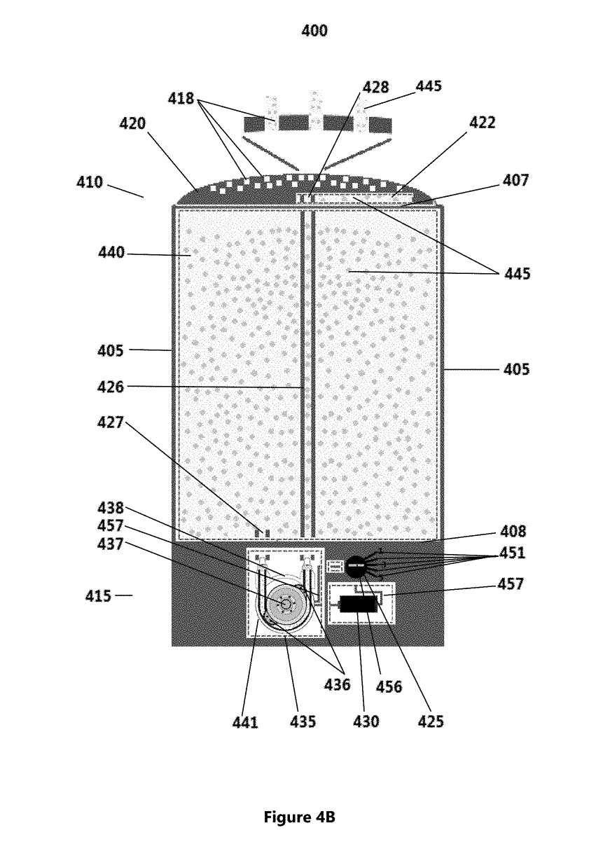

FIG. 4A illustrates a front view of an embodiment of a dispensing device 400 according to the present invention, with sidewall cutouts indicated by dashed lines. Dispenser device 400 possesses sidewalls 405, partition walls 407 and 408, application end 410, and dispensing end 415. Dispenser device 400 is sized and shaped for facile and comfortable hand held use. Sidewalls 405 are integral with each of application end 410 and dispensing end 415. Application end 410 comprises convex, apertured 418 application surface 420. Dispensing end 415 possesses actuator button 425, battery 430, and peristaltic pump 435.

Partition walls 407 and 408 sealingly adjoin sidewalls 405 at application end 410 and dispensing end 415, respectively. Disposed within sidewalls 405, partition wall 407, and partition wall 408, is storage reservoir 440 loaded with semi-solid, topical, pharmaceutical composition 445 that has a viscosity of from about 380 cPs to about 700 cPs, preferably from about 400 Cps to about 680 Cps at about room temperature. Disposed within application surface 420, sidewalls 405, and partition wall 407 is application end reservoir 422. Tube 426 places storage reservoir 440 and application end reservoir 422 in semi-solid gel communication. First end 427 of tube 426 is open and positioned in storage reservoir 440; and second end 428 of tube 426 is open and positioned in application end reservoir 422. From first open end 427, tube 426 sealingly passes through: partition wall 408, peristaltic pump 435, partition wall 408 a second time, storage reservoir 440, and partition wall 407.

Peristaltic pump 435 comprises spring loaded rollers 436, housing 441, drive shaft 437, and electric motor 438. The section of tube 426 that passes through peristaltic pump 435 is compressible against housing 441 by spring loaded rollers 436. Motor 438 rotates, counterclockwise in the illustrated perspective, drive shaft 437, together with spring loaded rollers 436 mounted thereon. Such that pharmaceutical composition 445 is pumped from storage reservoir 440 through tube 426 into application end reservoir 422 and to application surface 420 through application surface apertures 418 by the running of running motor 438.

Motor 438, battery 430, and actuator button 425 are in an electric circuit connected by wires 457. Motor 438 is powered by battery 430 in response to depression of programmable actuation button 425, which is rotatably adjustable, by turning slot 456 with a slot driver instrument (e.g., a screwdriver, coin, or the like) such that slot 456 points to one of five settings marked by numbered lines 451. Setting actuation button 425 to each incrementally larger setting results in peristaltic pump 435 pumping a greater, predetermined amount of pharmaceutical composition 445 from storage reservoir 422 to application surface 420 by, e.g., causing motor 438 to run faster or for a longer period of time per actuation.

The compressibility and inner diameter of the section of tube 426 compressed against housing 441 by rollers 436 is selected in conjunction with the gap between rollers 436 and housing 441, the circumference of the circle defined by the arced path along which rollers 426 travel and the rate and duration of motor 438 run time, and the viscosity of pharmaceutical composition 445 such that one actuation of peristaltic pump 435 results in a particular volume of pharmaceutical composition 445, ranging from about 50 microliters to about 15,000 microliters being extruded through apertures 418. FIG. 4B shows device 400 after one actuation.

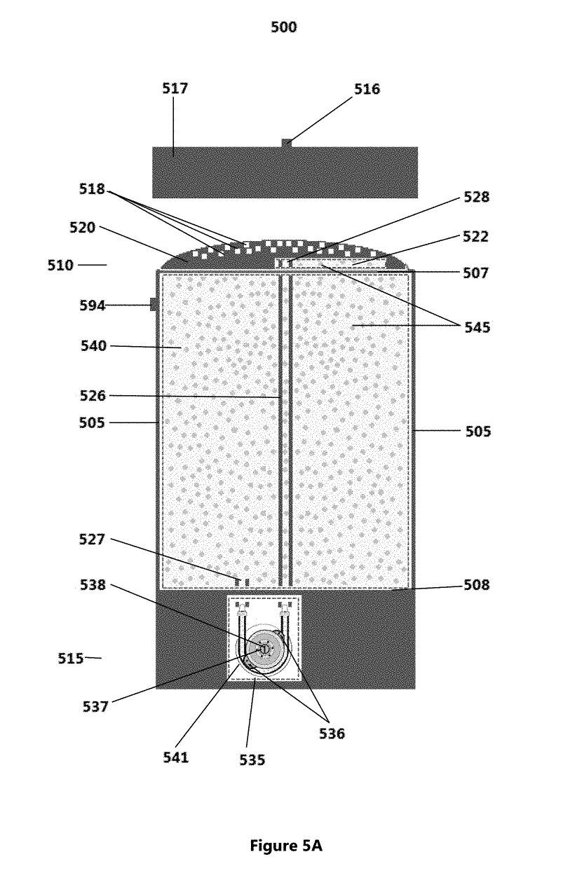

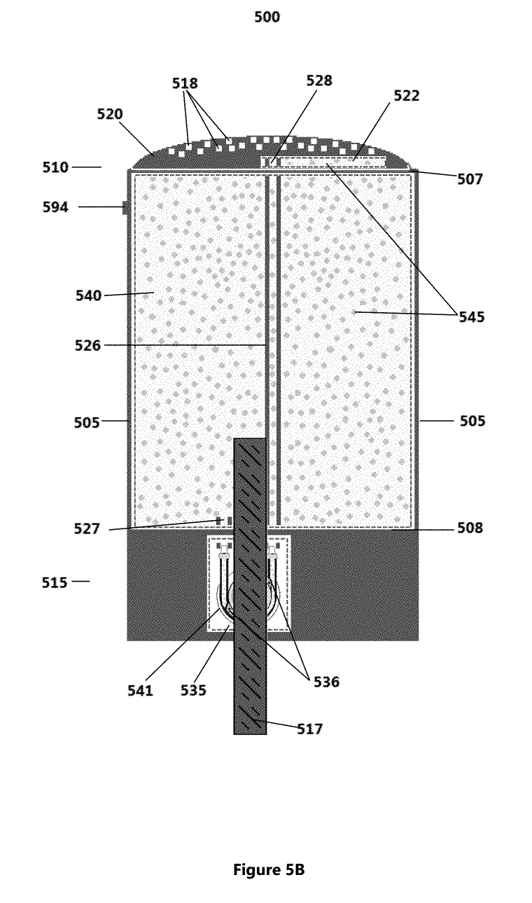

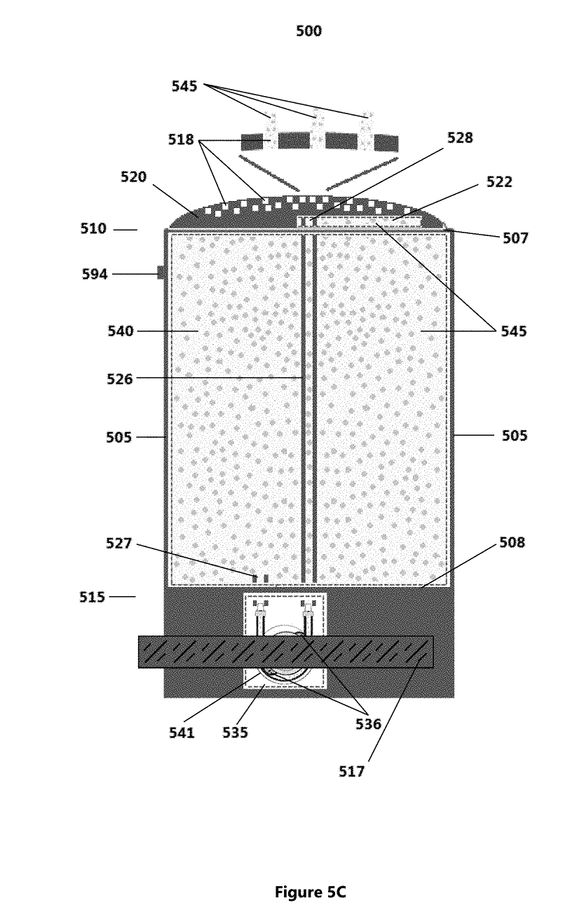

FIG. 5A illustrates an embodiment of a dispensing device 500 according to the present invention that differs from the dispenser device illustrated in FIG. 4 by possessing a crank peristaltic pump, with sidewall cutouts indicated by dashed lines. Dispenser device 500 possesses sidewalls 505, partition walls 507 and 508, application end 510, dispensing end 515, and removable key cap 517. Dispenser device 500 is sized and shaped for facile and comfortable hand held use. Sidewalls 505 are integral with each of application end 510 and dispensing end 515; and removable cap 517 is sized and shaped for frictional securement with the sidewalls 505 at application end 510. Application end 510 comprises convex, apertured 518 application surface 520. Dispensing end 515 possesses crank operated peristaltic pump 535.

Partition walls 507 and 508 sealingly adjoin sidewalls 505 at application end 510 and dispensing end 515, respectively. Disposed within sidewalls 505, partition wall 507, and partition wall 508, is storage reservoir 540 loaded with semi-solid, topical, pharmaceutical composition 545 that has a viscosity of from about 380 cPs to about 700 cPs, preferably from about 400 Cps to about 680 Cps at about room temperature. Disposed within application surface 520, sidewalls 505, and partition wall 507 is application end reservoir 522. Tube 526 places storage reservoir 540 and application end reservoir 522 in semi-solid gel communication. First end 527 of tube 526 is open and positioned in storage reservoir 540; and second end 528 of tube 526 is open and positioned in application end reservoir 522. From first open end 527, tube 526 sealingly passes through: partition wall 508, peristaltic pump 535, partition wall 508 a second time, storage reservoir 540, and partition wall 507.

Peristaltic pump 535 comprises spring loaded rollers 536, housing 541, drive shaft 537, which possesses crank slot 538. The section of tube 526 that passes through peristaltic pump 525 is compressible against housing 541 by spring loaded rollers 536. Counterclockwise cranking of slot 538 with a slot driver instrument (e.g., slot key 516 of key cap 517, a screwdriver, a coin, or the like) results in the counterclockwise rotation of drive shaft 537, together with spring loaded rollers 536 mounted thereon. Such that pharmaceutical composition 545 is pumped from storage reservoir 520 through tube 526 into application end reservoir 522 and to application surface 520 through application surface apertures 518.

The compressibility and inner diameter of tube section 539 is selected in conjunction with the gap between rollers 536 and housing 541, the circumference of the circle defined by the arced path along which rollers 526 travel, and the viscosity of pharmaceutical composition 545 such that one actuation of peristaltic pump 535, which corresponds to cranking drive shaft 537 such that it rotates from of from about 10° to about 150°) results in a particular volume of pharmaceutical composition 545, ranging from about 50 microliters to about 15,000 microliters being extruded through apertures 518. FIG. 5B shows device 500 with slot key 516 of positioned in crank slot 538. key FIG. 5C shows device 500 after one actuation: a 90°, counterclockwise rotation of cap 517, slot key 516, and drive shaft 537. Pressure release butter fly valve 594 is operative to relieve negative pressure from reservoir 540 after a plurality of uses of device 500 deplete pharmaceutical formulation 545 from reservoir 540.

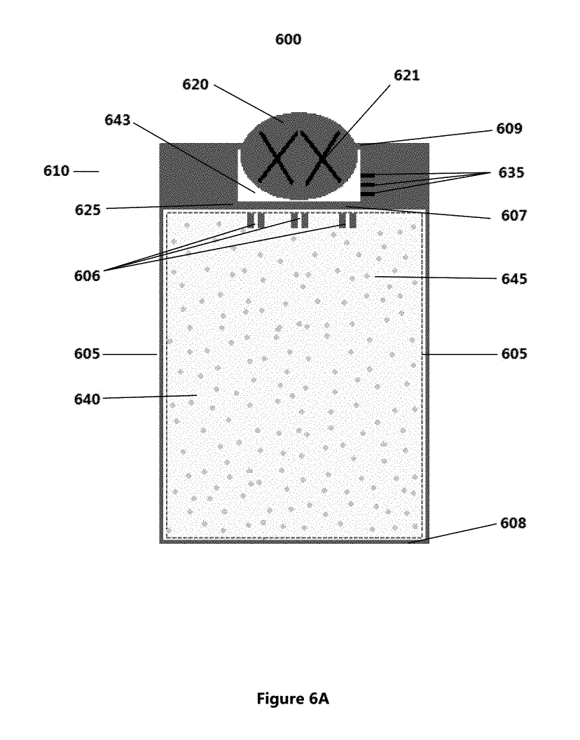

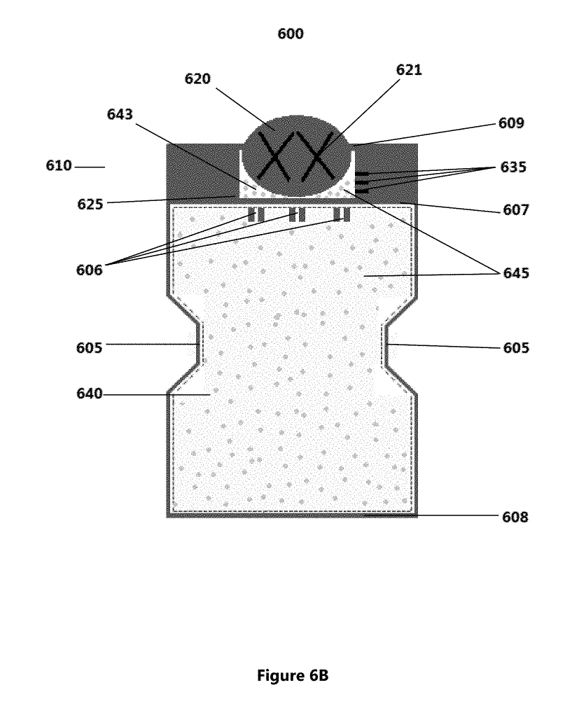

FIG. 6A illustrates a front view of an embodiment of a dispensing device 600 according to the present invention, with sidewall cutouts indicated by dashed lines. Dispenser device 600 possesses compressible sidewalls 605, partition wall 607, end walls 608 and 609, and application end 610. Dispenser device 600 is sized and shaped for facile and comfortable hand held use. Sidewalls 605 are integral with application end 610. Application end 610 comprises partition wall 607, window 625, application roller ball 620, and graduation lines 635.

Sidewalls 605 sealingly adjoin each of partition wall 607 and endwalls 608 and 609. Disposed within sidewalls 605, partition wall 607, and end wall 608, is storage reservoir 640 loaded with semi-solid, topical, pharmaceutical composition 645 that has a specific gravity of from about 0.85 to about 1.20, preferably about 0.90 to about 1.15 at about room temperature. Disposed within sidewalls 605, partition wall 607, and end wall 609, is application reservoir 643. One way openings 606 in partition wall 607 allow one-way flow of pharmaceutical composition 645 from storage reservoir 640 to application reservoir 643 upon mechanical compression (e.g., by hand squeezing) of side walls 605 (FIG. 6B).

Graduation lines 635 allow for the transfer of measured amounts of pharmaceutical composition 645 from storage reservoir 640 to application reservoir 643. Graduation lines 635 allow for reliable metering into application reservoir 643 amounts of pharmaceutical composition 645, typically ranging from about 50 microliters to about 15,000 microliters.

Application roller ball 620 possesses knurled surface 621 and is sized, shaped, and spinnably positioned such that less than half of its volume protrudes through the opening therefor in endwall 608 and more than half of its volume is disposed within application reservoir 643. Contacting roller ball 620 with skin with moderate pressure, together with movement of device 600, along a path substantially parallel to the surface of the skin, causes the spinning of roller ball 620 and efficient transfer of pharmaceutical formulation 645 from application reservoir 643 to application surface 621 and then to the skin.

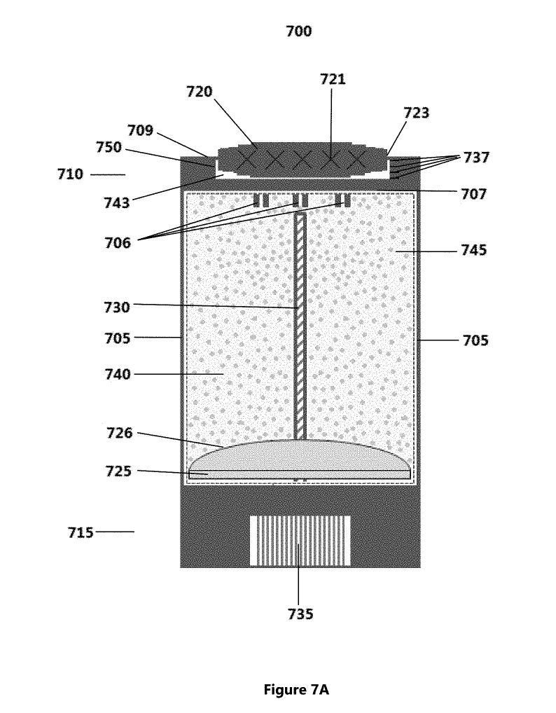

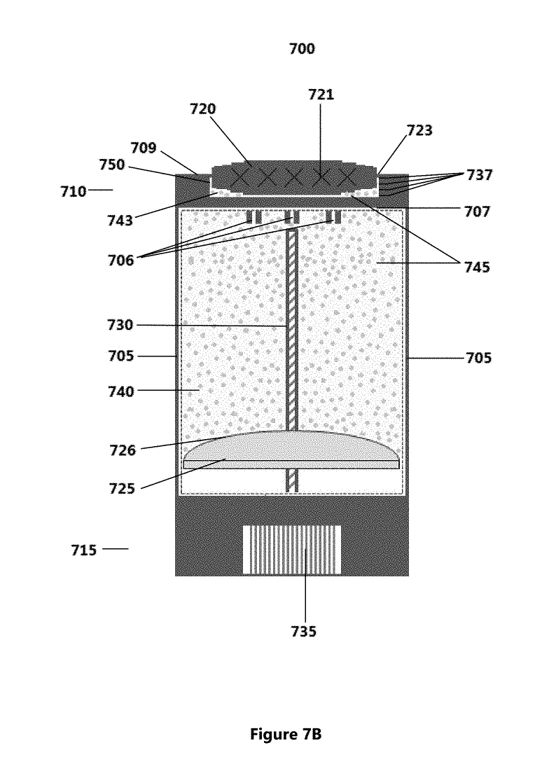

FIG. 7A illustrates a front view of an embodiment of a dispensing device 700 according to the present invention, with sidewall cutouts indicated by dashed lines. Dispenser device 700 possesses sidewalls 705, partition wall 707, dispenser end 715, and application end 710. Dispenser device 700 is sized and shaped for facile and comfortable hand held use. Sidewalls 705 are integral with each of application end 710 and dispensing end 715.

Disposed within sidewalls 705 at dispensing end 715 is a dispensing assembly that includes platform 725, threaded shaft 730, and actuator dial 735. Disposed within sidewalls 705, partition wall 707, and platform 725, is volumetrically collapsible reservoir 740 loaded with semi-solid, topical, pharmaceutical composition 745 that has a specific gravity of from about 0.85 to about 1.20, preferably about 0.90 to about 1.15 at about room temperature. Actuator dial 735 is touch accessible through openings 755 in sidewalls 705.

Platform 725 has convex surface 726 and is in circumferentially wiping contact with inner surfaces of sidewalls 705. Platform 725 is threadedly secured to drive shaft 730, which is fixedly secured to actuator dial 735 substantially at the center thereof. The thread pitch of threaded shaft 730 causes an advancing action of actuator dial 735 to result in the movement of platform 725 toward partition wall 707 (FIG. 7B).

Application end 710 is comprised of partition wall 707, end wall 709, window 750, application roller bar 720, roller bar axle 723, and graduation lines 737. Sidewalls 705 sealingly adjoin each of partition wall 707 and endwall 709. Disposed within sidewalls 705, partition wall 707, and end wall 709, is application reservoir 743.

One way openings 706 in partition wall 707 allow one-way flow of pharmaceutical composition 745 from storage reservoir 740 to application reservoir 743 upon volumetric compression (e.g., by an actuating advance of actuator dial 735) of storage reservoir 740 (FIG. 7B). Graduation lines 737 allow for the transfer of measured amounts of pharmaceutical composition 745 from storage reservoir 740 to application reservoir 743. Graduation lines 737 allow for reliable metering into application reservoir 743 amounts of pharmaceutical composition 745, typically ranging from about 50 microliters to about 15,000 microliters.

Application roller bar 720 possesses knurled surface 721 and is rotatably mounted on axle 723, which is fixedly mounted on endwall 709. Contacting roller bar 720 with skin with moderate to high pressure, together with movement of device 700, along a path substantially parallel to the surface of the skin, results in efficient transfer of pharmaceutical formulation 745 from application reservoir 743 to application surface 721 and then to the skin.

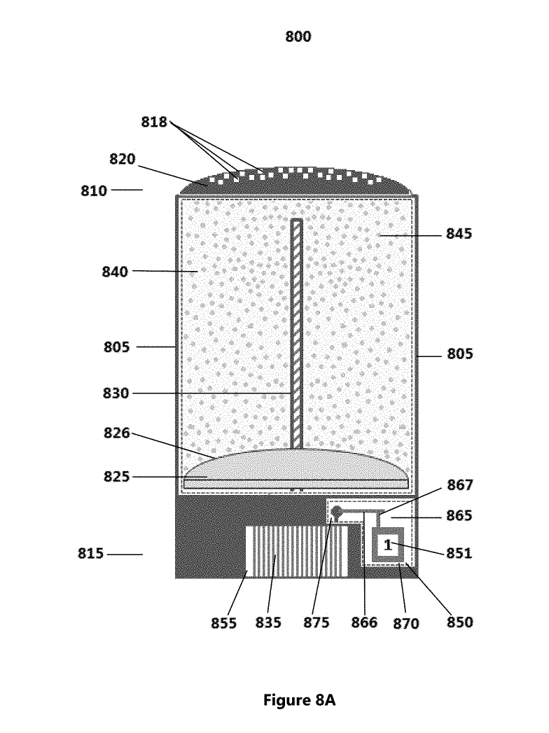

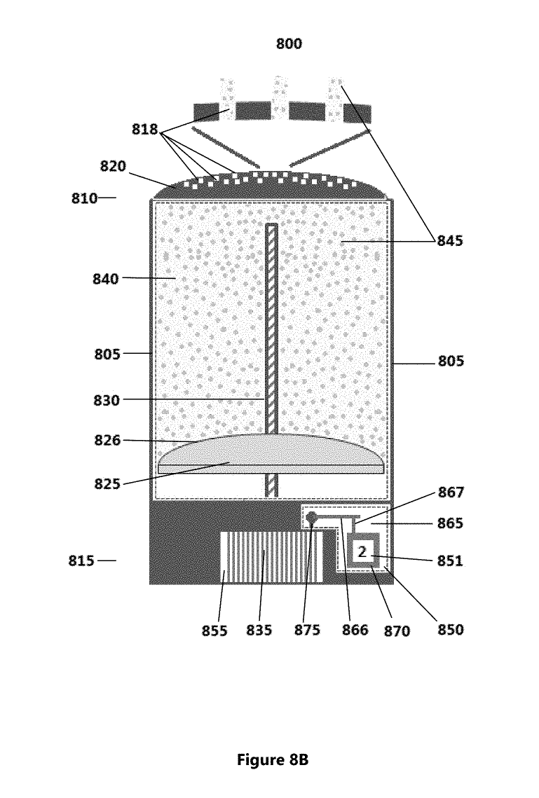

FIG. 8A illustrates a front view of an embodiment of a semi-solid, topical, pharmaceutical composition dispensing device 800 according to the present invention, with sidewall cutouts indicated by dashed lines. Dispenser device 800 differs from the device illustrated in FIG. 1 by possessing mechanical dose count indicator display unit 850, rather than an electronic dose indicator.

Dispenser device 800 possesses sidewalls 805, application end 810, and dispensing end. Dispenser device 800 is sized and shaped for facile and comfortable hand held use. Sidewalls 805 are integral with each of application end 810 and dispensing end 815. Application end 810 comprises convex, apertured 818 application surface 820. Disposed within sidewalls 805 at dispensing end 815 is a dispensing assembly that includes platform 825, threaded shaft 830, and actuator dial 835. Disposed within sidewalls 805, application surface 820, and platform 825, is volumetrically collapsible reservoir 840 loaded with semi-solid, topical, pharmaceutical composition 845 that has a specific gravity of from about 0.85 to about 1.20, preferably about 0.90 to about 1.15 at about room temperature. Actuator dial 835 is touch accessible through opening 855 in sidewalls 805.

Platform 825 has convex surface 826 and is in circumferentially wiping contact with inner surfaces of sidewalls 805. Platform 825 is threadedly secured to drive shaft 830, which is fixedly secured to actuator dial 835 substantially at the center thereof. The thread pitch of threaded shaft 830 is selected in conjunction with the diameter of actuator dial 835, the size of the apertures 818, and the specific gravity of pharmaceutical composition 845 so that one advancing action of actuator dial 835 (to produce a shaft 830 rotation of from about 10° to about 150°) advances platform 825 a distance toward application surface 820 sufficient to result in a particular volume of pharmaceutical composition 845, ranging from about 50 microliters to about 15,000 microliters, being extruded through apertures 818 (FIG. 8B). Accordingly, rubbing application surface 820 against skin after one, two, three, etc. advancing actions transfers to the skin a metered amount of extruded pharmaceutical composition 845

Dose count display unit 850 possesses pivot switch 865 and housing 870 fixedly mounted on sidewall 805. Fixedly mounted on actuator dial 835 are a plurality of ball and stick dose counter triggers 875 (one shown). Dose counter triggers 875 are positioned along the circumference of a circle that is concentric with, and has a smaller radius than, actuator dial 835.

Pivot switch 865 comprises deflection bar 866 fixedly secured to the top of switch pin 867, which is rotatably mounted into housing 870. Pivot switch 865 is tension mounted in the illustrated position. Toward the end of one advancing action of actuator dial 835, movement of a dose counter trigger 875 along its arced path, caused by finger advancing actuator dial 835, results in forceful contact by the ball portion of dose counter trigger 875 against deflection bar 866. Such contact causes deflection bar 866 to deflect out of the path of dose counter trigger 875, which in turn causes axial rotation of switch pin 867. At the end of one advancing action of actuator dial 835, dose counter trigger 875 moves further along its arced path out of contact with deflection bar 866, which allows pivot switch 865 to return to its tension mounted position. At the point of maximal rotation of switch pin 867, achieved at the point of maximum deflection of deflection bar 866 away from its tension mounted position, the rotation of switch pin 866 causes the transmission of an mechanical signal that results in dose counter display unit 850 advancing dose count readout 851 to reflect the occurrence of an actuation event (FIG. 8B).

Application surfaces of devices according to the present invention are sized and shaped for comfortable and efficient application of pharmaceutical compositions to target skin surface regions of the human body including, without limitation, forehead, glabella, face, cheek, neck, shoulder, sternum, manubrium, axilla, hand, palm, finger, interdigital web, abdominal, buttock, leg, thigh, shin, calf, ankle, knee, volar, toe, and toe web regions.

Medicaments.

As used herein “medicament” refers to drugs, pharmaceutical substances, active pharmaceutical ingredients, therapeutic agent, bioactive agents, and the like. Medicaments can comprise organic or inorganic compounds or molecules, nucleic acids, polypeptides, peptides, proteins, saccharides, lipids, and the like. Medicaments can fall under a variety of biological activity classes, including without limitation steroid and non-steroidal anti-inflammatory drugs, analgesics, anesthetics, antibiotics, antimicrobials, tranquilizers, sedatives, narcotics, antihistaminics, antifungals, antivirals, disinfectants, antipsoriasis agents, local anesthetics, vasoactive agents, neuroactive agents, hormones, steroids, anticoagulants, antipruritics, immunomodulating agents, cytotoxic agents, anticancer agents, hormone replacement agents, antigens, and antibodies.

Specific medicaments that can be incorporated into pharmaceutical compositions dispensed by devices of the present invention include, without limitation: anesthetics such as benzocaine, procaine hydrochloride, cocaine, novocaine, xylocaine, tetracaine, tetracaine hydrochloride, dibucaine, lidocaine, lidocaine hydrochloride, bupivicaine, dyclonin, etidocaine, mepivicaine, butamen picrate, dimethisoquin hydrochloride, cyclomethylcaine sulfate, and the like; analgesics and anti-inflammatory agents such as buprenorphin, butophanol tartrate, acetaminophen, fentanyl, mefenamic acid, flutenamic acid, diclofenac, oxyphenbutazone, phenybutazone, ibuprofen, flurbiprofen, naproxen, menthol, methyl salicylate, phenol, salicylic acid, benzyl alcohol, camphor, camphorated metacresol, juniper tar, resorcinol, allyl isothiocyanate, capsaicin, and the like; corticosteroids such as alclometasone dipropionate, amcinocide, hydrocortisone, betamethasone dipropionate, betamethasone valerate, desoximetasone, clobetasol propionate, flurandrenolide, halcinonide, halobetasol, estradiol, testosterone, progesterone, fluticasone, clobetasol, dexamethasone, dexonide, fluocinolone acetonide, flucinonide, medroxyprogesterone, mometasone furoate, triamcinolone, and the like; hormones (such as estrogens, estradiol, progesterol, progesterone, testosterone, insulin, calcitonin, parathyroid hormone, peptide and vasopressin hypothalamus releasing factor); antibiotics such as β-lactams (e.g., amoxicillin, ampicillin, bacampicillin, carbenicillin, cloxacillin, dicloxacillin, flucloxacillin, methicillin, mezlocillin, nafcillin, oxacillin, penicillin G, penicillin V, piperacillin, pivampicillin, pivmecillinam, ticarcillin, sulbactam, tazobactam, clavulanate), cephalosporins (e.g., cefaclor, cefadroxil, cefamandole, cefazolin, cefdinir, cefditoren, cefepime, cefixime, cefonicid, cefoperazone, cefotaxime, cefotetan, cefoxitin, cefpodoxime, cefprozil, ceftazidime, ceftibuten, ceftizoxime, ceftriaxone, cefuroxime, cephalexin, cephalothin, cephapirin, cephradine), aminoglycosides (e.g., gentamycin, streptomycin, amikacin, kanamycin, viomycin, capreomycin), ethionamide, prothionamide, cycloserine, dapsone, clofazimine, tetracyclines (e.g., tetracycline, doxycycline, chlortetracycline, oxytetracycline, minocycline demeclocycline), oxazolidinones (e.g., linezolid, eperezolid), quinolones (e.g., cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, rulifloxacin, delafloxacin, and nemonoxacin) metronidazole, rifabutin, isoniazonid, and ethambutol) bacitracin, chlorhexadine gluconate, clindamycin, cliquinol, neomycin sulfate, polymyxin B sulfate, erythromycin, gentamicin, sulfathiazole, sulfacetamide, sulfabenzamide, oxytetracycline hydrochloride, and the like; antimicrobial agents such as benzalkonium chloride, chlorhexidine gluconate, hexachlorophene, mafenide acetate, nitrofurazone, nystatin, acetosulfamine, clortrimazole, povidone-iodine, and the like; antifungal agents such as amphotericin B, butoconazole, cetylpyridinium chloride, chlorxylenol, cyclopirox olamine, clioquinol, clotrimazole, sulconazole nitrate, nystatin, oxyconazole, econazole nitrate, ketoconazole, miconazole nitrate, naftifine hydrochloride, pentamycin, pyrrolinitrin, terbinafine, triacetin, and the like; debriding agents such as deoxyribonuclease, collagenolytic, debridement, fibrinolytic or proteolytic enzymes, papain, papain-urea, and the like; antihistamines such as chlorcyclizine hydrochloride, diphenylhydramine hydrochloride, tripelennamine hydrochloride, and the like; antiepileptics such as nitrazepam, meprobamate, clonazepam, and the like; coronary vasodilators such as nitroglycerine, dipyridamole, erythritol, tetranitrate, pentaerythritol tetranitrate, propatyinitrate, and the like; antivirals such as amantadine, rimantadine, pleconaril, acyclovir, gancyclovir, zidovudine, lamivudine, RNase H inhibitors, integrase inhibitors, protease inhibitors, rifampicin, zanamivir, oseltamivir, and interferons, and the like; dermatologicals such as retinal, retinol, retinoic acid and their derivatives, hydroxyacids, alphaketoacids, and the like; and other drugs such as benzoyl peroxide, podofilox, masoprocol, nicotine, scopolamine, nitroglycerine, fluorouracil, hydrocolloids, hydroquinone, monobenzone, tretinoin and acyclovir; and combinations thereof. Other medicaments useful in the present invention include those described in U.S. Pat. No. 8,343,962, the content of which is hereby incorporated by reference in its entirety.

Useful amounts of medicaments in pharmaceutical compositions of the invention include 0.001% w/w, 0.05% w/w, 0.1% w/w, 0.25% w/w, 0.5% w/w, 0.75% w/w, 1.0% w/w, 2.5% w/w, 5.0% w/w, 7.5% w/w, 10% w/w, 12.5% w/w, 15% w/w, 20% w/w, 25% w/w, 30% w/w, 40% w/w, 50% w/w, and ranges therebetween.

Thickening Agents.

The thickening agents that can be incorporated into pharmaceutical compositions dispensed by devices of the present invention include: celluloses (e.g., microcrystalline cellulose, methylcellulose, ethylcellulose, carboxymethyl cellulose, and carboxyethyl cellulose), high molecular weight polymers of acrylic acid crosslinked with alkyl esters of sucrose or pentaerythritol (e.g., carbomer 910, carbomer 934, carbomer 940, carbomer 941, carbomer 971, carbomer 974, carbomer 980, carbomer 981, and carbomer 1342); hyaluronic acid; glycosaminoglycanes (e.g., heparin and chondroitin sulfate); polyethylene glycol, fucoidan; polyamino acids (e.g, poly-aspartic acid and poly-glutamic acid); alginates; pectins; gellan, carboxyalkyl chitins; carboxymethyl chitosans; sulfated polysaccharides; glycosaminoglycanes, polylactates; acrylic acid polymers crosslinked with divinyl glycol (e.g., polycarbophils); poloxamers; tyloxapols; silicone polymers; and mixtures thereof.

Useful amounts of thickening agents in pharmaceutical compositions of the invention include 0.001% w/w, 0.05% w/w, 0.1% w/w, 0.25% w/w, 0.5% w/w, 0.75% w/w, 1.0% w/w, 2.5% w/w, 5.0% w/w, 7.5% w/w, 10% w/w, 12.5% w/w, 15% w/w, 20% w/w, and ranges therebetween.

Solvents.

Solvents that can be incorporated into pharmaceutical compositions dispensed by devices of the invention include, without limitation, polar and nonpolar solvents such as alcohols, propylene glycol, glycerol, ethers, polyethylene glycols, amides; esters, water, and other solvents known in the art.

Skin Penetration Enhancers.

Skin penetration enhancers that can be incorporated into pharmaceutical compositions dispensed by devices of the present invention include: glyceryl oleate (glycerol monooleate); isopropyl myristate; methyl laurate; N-lauroyl sarcosine; oleic acid (octadecenoic acid); sodium lauryl sulfoacetate; sodium octyl sulfate; and mixtures thereof.

Useful amounts of skin penetration enhancers in pharmaceutical compositions of the invention include 0.001% w/w, 0.05% w/w, 0.1% w/w, 0.25% w/w, 0.5% w/w, 0.75% w/w, 1.0% w/w, 2.5% w/w, 5.0% w/w, 7.5% w/w, 10% w/w, 12.5% w/w, 15% w/w, 20% w/w, 25% w/w, and ranges therebetween.

Antiperspirants and Deodorants.

Antiperspirants and deodorants that can be incorporated into pharmaceutical compositions dispensed by devices of the present invention include, without limitation, aluminum, zinc, or zirconium salts and complexes, such as halides, hydroxy halides, activated chlorohydrates (e.g., aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like), and mixtures thereof.

Useful amounts of antiperspirants and deodorants in pharmaceutical compositions of the invention include 0.001% w/w, 0.05% w/w, 0.1% w/w, 0.25% w/w, 0.5% w/w, 0.75% w/w, 1.0% w/w, 2.5% w/w, 5.0% w/w, 7.5% w/w, 10% w/w, 12.5% w/w, 15% w/w, 20% w/w, and ranges therebetween.

Surfactants.

Surfactants that can be incorporated into pharmaceutical compositions dispensed by devices of the invention include, without limitation, ionic, nonionic, and zwitterionic surfactants such as alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, alkyl oligoglucoside sulfates, protein fatty acid condensates, alkyl (ether) phosphates, fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, glucuronic acid derivatives, fatty acid-N-alkyl glucamides, protein hydrolyzates, polyol fatty acid esters, sugar esters, sorbitan esters, polysorbates, amine oxides, dimethyl distearyl ammonium chloride, esterquats, alkylbetaines, alkylamidobetaines, aminopropionates, aminoglycinates, imidazoliniumbetaines and sulfobetaines, fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals, and protein fatty acid condensates.

Useful amounts of surfactants in pharmaceutical compositions of the invention include 0.0001% w/w, 0.001% w/w, 0.05% w/w, 0.1% w/w, 0.25% w/w, 0.5% w/w, 0.75% w/w, 1.0% w/w, 2.5% w/w, 5.0% w/w, 7.5% w/w, 10% w/w, and ranges therebetween.

Oils.

Surfactants that can be incorporated into pharmaceutical compositions dispensed by devices of the invention include, without limitation,

Useful amounts of oils in pharmaceutical compositions of the invention include 0.001% w/w, 0.05% w/w, 0.1% w/w, 0.25% w/w, 0.5% w/w, 0.75% w/w, 1.0% w/w, 2.5% w/w, 5.0% w/w, 7.5% w/w, 10% w/w, 12.5% w/w, 15% w/w, 20% w/w, 25% w/w, 30% w/w, 40% w/w, 50% w/w, 60% w/w, 70% w/w, 80% w/w, 90% w/w, and ranges therebetween.

While particular embodiments of the invention have been illustrated and described, various modifications will be apparent to those skilled in the art. For example, several pharmaceutical compositions illustrated in the figures appear to have particles in suspension, the scope of the pharmaceutical compositions useful in the present invention is not limited to suspensions, but extends to solution compositions. Accordingly, it is not intended that the invention be limited to the disclosed embodiments or to details thereof, and departures may be made therefrom within the spirit and scope of the invention. In addition, the scope of the present invention extends to devices that comprise components illustrated separately in different figures, but mixed and matched into a single device.

What is claimed is:

1. A hand-held device, for applying a metered amount of a topical pharmaceutical composition to a dermal surface of a patient, the hand-held device comprising a reservoir for the topical pharmaceutical composition, an applicator surface adapted for administering the topical pharmaceutical composition to the dermal surface, and means for dispensing a metered amount of the topical pharmaceutical composition from the reservoir to the applicator surface, wherein:
the topical pharmaceutical composition is a nonpourable gel and comprises a medicament, a thickening agent, and a solvent,
the reservoir is:
i. sized and shaped for containing the topical pharmaceutical composition within the hand-held device;
ii. formed from, or lined with, a material that is at least one member selected from the group consisting of: opaque and substantially nondepleting of the medicament;
iii. adapted for transmission of the topical pharmaceutical composition from the reservoir to the applicator surface; and
iii. fitted, on a first side or end of the reservoir and in a volumetrically compressible manner, with the means for dispensing the metered amount of the topical pharmaceutical composition, and
the means for dispensing a metered amount of the topical pharmaceutical composition comprises a platform that possesses a hole sized to slideably receive a threaded shaft of uniform pitch that is in fixed connection to an actuator that is rotatably mounted to the hand-held device, such that rotation of the actuator by a particular number of degrees results in: a. the platform moving perpendicularly upwards along the shaft a corresponding particular distance that volumetrically compresses the reservoir, and b. the transmission of the metered amount of the topical pharmaceutical composition from the reservoir to the applicator surface.

2. The device of claim 1, wherein the medicament is present in the topical pharmaceutical composition in an amount of from 0.001% w/w to 7.5% w/w, and wherein the medicament is selected from the group consisting of an estrogen and a testosterone.

3. The device of claim 2, wherein the thickening agent is present in the topical pharmaceutical composition in amount of from 0.001% w/w to 10.0% w/w, and wherein the thickening agent is at least one member selected from the group consisting of a celluloses, a carbomer; a hyaluronic acid; a glycosaminoglycanes; a polyethylene glycol, a fucoidan; an alginate; a pectin; a gellan, a polylactate; a poloxamer; a tyloxapol; and a silicone polymer.

4. The device of claim 3, wherein the solvent is at least one member selected from the group consisting of an alcohol, a propylene glycol, a glycerol, an ether, a polyethylene glycol, an amide; an esters, and water.

5. The device of claim 4, wherein the topical pharmaceutical composition further comprises a skin penetration enhancer, and wherein the skin penetration enhancer is present in the topical pharmaceutical formulation in an amount of from 0.001% w/w to 12.5% w/w, and wherein the skin penetration enhancer is at least one member selected from the group consisting of a glyceryl oleate, an isopropyl myristate; a methyl laurate; a N-lauroyl sarcosine; an octadecenoic acid, a sodium lauryl sulfoacetate; and a sodium octyl sulfate.

6. The device of claim 5, wherein the topical pharmaceutical composition further comprises a deodorant, and wherein the deodorant is present in the composition in an amount of from 0.001% w/w to 2.5% w/w, and wherein the deodorant is at least one member selected from the group consisting of an aluminum dichlorohydrate, an aluminum-zirconium octachlorohydrate, an aluminum sesquichlorohydrate, an aluminum chlorohydrex propylene glycol, an aluminum dichlorohydrex propylene glycol, an aluminum sesquichlorohydrex propylene glycol, an aluminum chlorohydrex polyethylene glycol, an aluminum dichlorohydrex polyethylene glycol, an aluminum sesquichlorohydrex polyethylene glycol, an aluminum-zirconium trichlorohydrate, an aluminum zirconium tetrachlorohydrate, an aluminum zirconium pentachlorohydrate, an aluminum zirconium octachlorohydrate, an aluminum zirconium trichlorohydrex glycine complex, an aluminum zirconium tetrachlorohydrex glycine, aluminum zirconium pentachlorohydrex glycine complex, aluminum zirconium octachlorohydrex glycine, zirconium chlorohydrate, an aluminum chloride, and aluminum sulfate.

7. The device of claim 6, wherein the medicament is present in the topical pharmaceutical formulation in suspension.

8. The device of claim 6, wherein the medicament is present in the topical pharmaceutical formulation in solution.

9. The device of claim 6, wherein the topical pharmaceutical formulation possesses, at room temperature, a specific gravity in the range from about 0.85 to about 1.20.

10. The device of claim 6, wherein the particular number of degrees is 90 degrees.

11. The device of claim 6, wherein the actuator is a dial actuator.

12. The device of claim 6, wherein the actuator is a slot key actuator

13. The device of claim 6, further comprising a cap.

14. The device of claim 6, further comprising a dose counter.

15. The device of claim 6, wherein the metered amount of the pharmaceutical composition is between 50 microliters and 1,500 microliters.
